(19) United States
(12) Patent Application Publication
(10) Pub. No.: US 2023/0240329 A1
(43) Pub. Date: Aug. 3, 2023

(54) SYSTEM AND METHOD FOR PRODUCING BYPRODUCTS FROM SPENT GRAINS

(71) Applicant: TERRA BIOPLASTICS INC., Toronto (CA)

(72) Inventors: Steve GEORGE, Toronto (CA); Ricardo MARTINEZ-VILLEGAS, Toronto (CA)

(21) Appl. No.: 17/927,767

(22) PCT Filed: May 28, 2021
(86) PCT No.: PCT/CA2021/050724

§ 371 Date: Nov. 25, 2022

Related U.S. Application Data
(60) Provisional application No. 63/031,279, filed on May 28, 2020.

(51) Int. Cl. A23K 10/38; A23K 10/14

(52) U.S. Cl. CPC A23K 10/38; A23K 10/14

(57) ABSTRACT

A method and system for producing at least one byproduct from spent grains, such as Brewer's Spent Grains or Distiller's Spent Grains. In one embodiment, the spent grains are preserved via a preservation treatment before being put through a cellulase treatment. The cellulase treated spent grains are then filtered to produce at least one byproduct.

### CROSS-REFERENCE TO OTHER APPLICATIONS

The current disclosure claims the benefit of priority from U.S. Provisional Application No. 63/031,279 filed May 28, 2020, which is hereby incorporated by reference.

### FIELD

The disclosure is generally directed at the brewing industry, and more specifically, at a system and method for producing byproducts from spent grains.

### BACKGROUND

In the brewing industry, there is typically a lot of waste that exists once the brewing process is complete. This waste is typically known as brewer's spent grains or distiller's spent grains, seen collectively as spent grains. Currently, these spent grains are used in the production of feed for animals or in the generation of energy.

However, spent grains are a valuable source of materials that can be directly and indirectly used in human nutrition and as industrial feedstocks. The use of spent grains in human nutrition can result in food that has a far lower environmental footprint than most other plant or animal based foods. The use of spent grains may support the ongoing transition to a “green” and “circular” economy. The increased and more efficient utilization of spent grains also allow the brewing industry and distilling industries to dramatically decrease their environmental footprint that comes from waste disposal.

Therefore, there is provided a method and apparatus for producing byproducts from spent grains.

### SUMMARY

The disclosure is directed at a method and system for producing byproducts from spent grain. In one embodiment, the system disclosure is directed at the processing of brewer's spent grain or distiller's spent grain to produce at least one byproduct. In one embodiment, the at least one byproduct may include, but is not limited to, fermentable sugars, protein biomass, fats and fat derivatives.

In one aspect of the disclosure, there is provided a method of producing at least one byproduct from spent grains including applying a preservation treatment to the spent grains to produce a preserved spent grains mixture; applying a cellulase treatment to the preserved spent grains mixture to produce a cellulase treated spent grains mixture; and filtering the cellulase treated spent grains mixture to produce the at least one byproduct.

In another aspect, the disclosure further includes milling the preserved spent grains mixture to reduce a size of particles within the preserved spent grains mixture. In a further aspect, milling the preserved spent grains mixture includes wet milling or dry milling the preserved spent grains mixture. In yet another aspect, the disclosure includes wetting the spent grains before applying the preservation treatment.

In a further aspect, applying a preservation treatment includes applying an alkaline treatment to the spent grains. In yet another aspect, applying an alkaline treatment includes applying an alkaline food grade reagent to the spent grains. In an aspect, the alkaline food grade reagent includes slaked lime, soda ash or caustic soda with a pH>10. In a further aspect, applying a preservation treatment includes treating the spent grains with a high temperature steam or high temperature water. In yet a further aspect, applying an alkaline treatment occurs after applying the preservation treatment. In a further aspect, applying a preservation treatment includes applying a preservative to the spent grains. In another aspect, the preservative includes sodium or potassium metabisulfite or sorbate.

In yet another aspect, applying a cellulase treatment includes applying a cellulase to the preserved spent grains mixture at a predetermined temperature and pH level for a predetermined period of time based on the cellulase. In another aspect, the cellulase is Cellic Ctec2 or Cellic CTec3. In yet a further aspect, filtering the cellulase treated spent grains mixture to produce the at least one byproduct includes filtering the cellulase treated spent grains into a filtrate and a retentate. In yet another aspect, the disclosure further includes applying an acid treatment to the filtrate. In an aspect, applying an acid treatment to the filtrate includes adding phosphoric acid or citric acid to the filtrate. In another aspect, the disclosure further includes processing the retentate to produce meal replacement products. In yet another aspect, the disclosure includes processing the filtrate to produce at least one of oils, fats, dyes, extracted protein or a dewatered biomass.

In another aspect of the disclosure, there is provided a system for producing at least one byproduct from spent grains including a preservation treatment apparatus for receiving the spent grains and for applying a preservative to the spent grains to produce preserved spent grains; a cellulase treatment apparatus for applying a cellulase treatment to the preserved spent grains to produce cellulase treated spent grains; and a set of filters for filtering the cellulase treated spent grains.

In another aspect, the system further includes a dry mill or wet mill for miling the preserved spent grains.

### BRIEF DESCRIPTION OF THE DRAWINGS

Embodiments of the present disclosure will now be described, by way of example only, with reference to the attached Figures.

FIG. 1 is a schematic diagram of the system in its operational environment;

FIG. 2 is a flowchart outlining a method of producing at least one byproduct from a spent grain;

FIG. 3 is a flowchart of an embodiment of producing at least one byproduct from Brewer's Spent Grain (BSG);

FIG. 4 is a flowchart of an embodiment of producing at least one byproduct from Wet Distiller's Spent Grain (DSG);

FIG. 5 is a flowchart of an embodiment of producing at least one byproduct from Dry DSG;

FIG. 6 is a flowchart of an embodiment of processing byproducts; and

FIG. 7 is a flowchart showing another embodiment of processing byproducts.

### DETAILED DESCRIPTION

The disclosure is directed at a method and system for producing byproducts from spent grains. Examples of spent grains include, but are not limited to, brewer's spent grains (BSG) or distiller's spent grains (DSG), either wet or dry. In one embodiment, the spent grains are processed to obtain at least one byproduct. Examples of byproducts include, but are not limited to fermentable sugars, a high protein mass, sugar syrup, fats and fat derivatives.

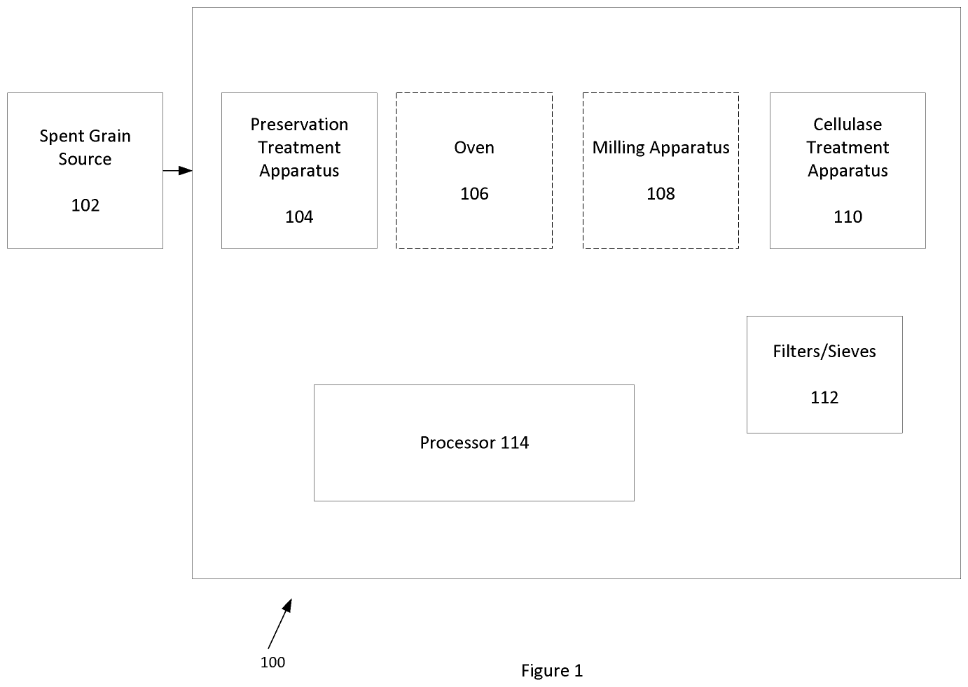

Turning to FIG. 1, a schematic diagram of an embodiment of the system is shown. The system 100 receives spent grains, such as BSG or DSG, from a source of spent grains 102. BSG may include spent barley or spent barley mixed with other grains while DSG may include spent corn, wheat, rice, or other grains. The system 100 includes a preservation treatment apparatus 104 that may be used to add a preservative to the spent grains. In one embodiment, the preservation treatment apparatus 104 may apply an alkaline pretreatment to the spent grains. In another embodiment, the preservation treatment apparatus 104 may be an apparatus that can apply high heat or high steam to the spent grains. In a further embodiment, the preservation treatment may apply ammonia or preservatives, such as, but not limited to, sodium or potassium sorbate or metabisulfate to the spent grains.

The system 100 may further include an oven 106 in embodiments where there is a need to dry out the spent grains either before or after it has received the preservation treatment. In embodiments where there is no need to dry the spent grains, the system may not include the oven 106. In other embodiments, the system 100 may also include a milling apparatus 108, such as a dry or wet milling apparatus, that is used to reduce a size of the spent grain particles.

The system 100 also includes a cellulase, or enzymatic hydrolysis, treatment apparatus 110 that is used to treat the spent grain mixture with cellulase at predetermined conditions in order to provide an enzymatic hydrolysis treatment to the spent grains. The system also includes a set of filters and sieves 112 to filter the cellulase treated spent grains in order to separate the spent grains into at least one byproduct. The system may further include a processor 114 that controls the different components of the system 100, such as to control the temperatures and/or speeds at which certain treatments are performed.

Although not shown, the system may also include other mechanical components that may be used to assist the passing of the spent grain between the different components of the system. For example, the system may also include a series of pumps to pump the spent grains through the set of filters and sieves. The system may also include a set of tanks that hold or store the spent grains as they are processed and treated. While the components may be distinct from each other, they may also be connected via a set of conveyors.

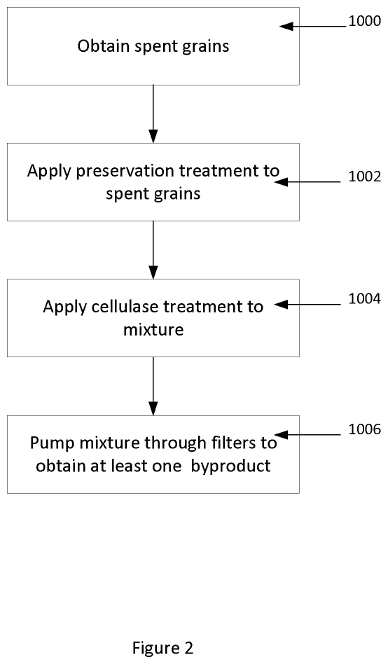

Turning to FIG. 2, a flowchart of one embodiment of producing at least one byproduct from spent grains is shown. Initially, spent grains are retrieved, or received, from a source of spent grains (1000). The spent grains may be BSG or DSG. The spent grains may be wet or dry when they are received whereby there may be a need to also dry or wet the spent grains. A preservation treatment is then applied to the spent grains (1002). In one embodiment, the preservation treatment may be an alkaline pre-treatment whereby a preservative is added to the spent grains. Other preservation treatments of the spent grains are contemplated and discussed below.

A cellulase treatment is then applied to the mixture (1004). After the cellulase treatment, the mixture is then pumped through sieves or filters (1006) to separate the mixture into at least one byproduct.

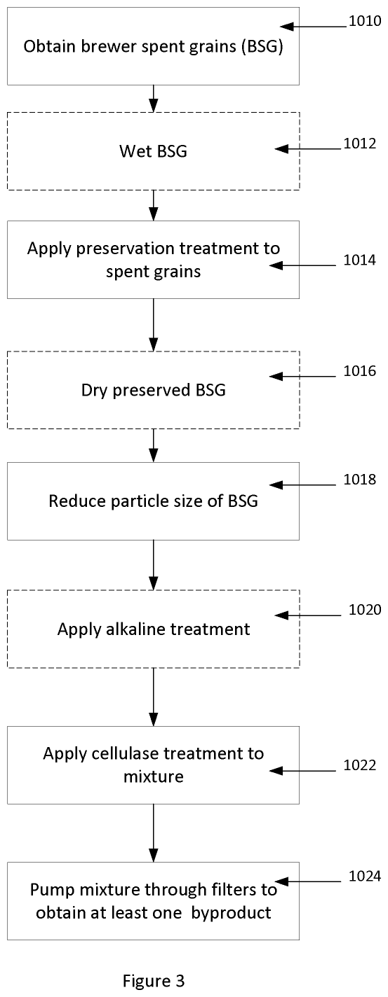

Turning to FIG. 3, a flowchart outlining one embodiment of a method of producing at least one byproduct from BSG is shown. The current embodiment may be seen as a process of fractionating BSG. In the current embodiment, the at least one byproduct being produced may include fermentable sugars (sugar syrup) and/or a high protein mass.

Initially, BSG is obtained from a source of BSG (1010). For the current method, the BSG is required to be wet, so the method may include wetting the BSG (1012) if it is obtained in dry form. The wet BSG is then subjected to a preservation treatment (1014) by adding, or applying, a preservative to the wet BSG. In one example, the preservative may be, but not limited to, a highly alkaline food grade reagent such as slaked lime (calcium hydroxide, Ca(OH)₂);a soda ash (sodium carbonate, Na₂CO₃, washing soda); caustic soda (sodium hydroxide, NaOH) with a pH>10; or a mild food grade acid such as, but not limited to, acetic acid or vinegar. In another embodiment, the preservative may be a mix of food grade preservatives, such as, but not limited to, sodium or potassium metabisulfite and, more specifically, sodium or potassium metabisulfite at a concentration >0.01% by weight. In yet a further embodiment, the preservative may be ammonia at a concentration of >0.1% by weight. In yet another embodiment, the preservation treatment may be an application of high temperature steam, including, but not limited to, autoclaving.

Depending on the preferred milling technique, the wet preserved BSG may then be dried, such as in an oven (1016).

The preserved BSG is then subjected to a particle size reduction (1018), such as via a milling technique. In one embodiment, if the preserved BSG is a wet BSG, this may be performed by using a wet milling technique. Wet milling techniques may include, but are not limited to, emulsion milling, disk milling or use of ball mills, cutter mills or roller mills. In an alternative embodiment, if the preserved BSG is a dry BSG, the dry BSG may be subjected to a dry milling technique. Dry milling techniques such as, but not limited to, disk mills, ball mills, cutter mills and roller mills may be used. Wet milling of wet preserved BSG results in a slurry being produced while dry milling of a dried preserved BSG forms a powder. In either embodiment, a BSG mixture having smaller particles is produced.

Depending on the type of preservation treatment, an alkaline treatment may then be applied to the milled BSG (1020). As discussed above, the alkaline treatment may include applying or adding a highly alkaline food grade reagent to the milled BSG. If the preservation treatment in (1014) was an alkaline treatment, there is no need to perform a further alkaline treatment.

This small particled BSG is then cellulase treated (1022) such as via enzyme hydrolysis using cellulases in a liquid. As the cellulase treatment is performed at specific ranges of temperature and pH for specific time intervals, the treatment may be performed in a stirred tank with temperature control, such as between 45 to 60° C. Examples of cellulases include, but are not limited to, Cellic Ctec2 or Cellic CTec3. In one embodiment, the cellulases may be added at a concentration of not less that 5 U per gram of wet biomass, and the reaction conducted at 45-60° C. and a pH of 4.5-5.5 for at least 24 hours. In some embodiments, further additives may be included in the treatment, such as, but not limited to, metal salts, or other enzymes such as laccases and surfactants.

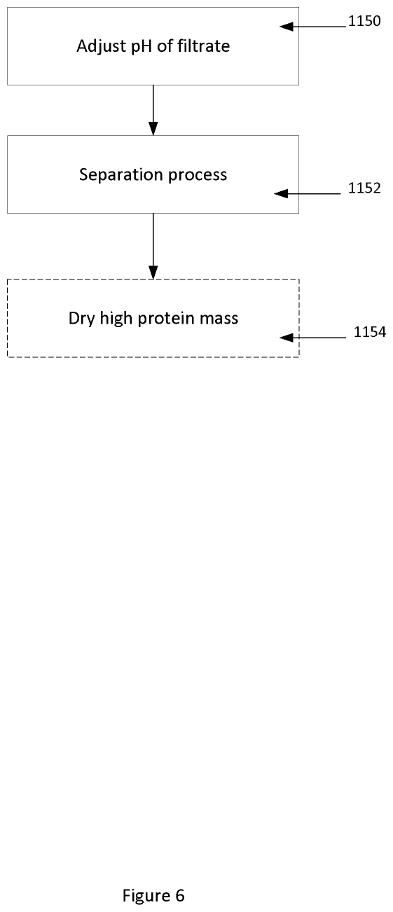

The mixture formed at the end of the enzymatic hydrolysis treatment is then pumped through sieves or filters (1024) to separate the at least one of the byproducts from each other. In the current embodiment, the byproducts of a sugar syrup and a residual high protein mass are separated from each other. In another embodiment, the sugar syrup may be separated from the protein mass by the use of progressively fine filtration methods such as a 500 μm filtration followed by a 20 μm filtration where the protein mass may be recovered from one or both of the filters. In one embodiment, the byproducts may be seen as a retentate and a filtrate where the filtrate may be a sugar syrup while the retentate may be a protein biomass. In another embodiment, the filtrate may be discarded and the retentate further processed to produce the at least one byproducts. One example of further processing, which may or may not be part of the overall process of producing at least one byproduct from spent grains, is described in more detail in FIG. 6.

In one specific example of the process of FIG. 3, 4000 g of a small particled preserved BSG was added to a tank with 20 L of drinking water. The solution was stirred with an impeller at 100 revolutions per minute (RPM) and the temperature raised to between 50° C.-60° C. The pH of the solution was then adjusted to within 4.5-5.5 with the use of a food grade acid. A commercial cellulase enzyme (Cellic CTec2) was then added to a concentration of 10 U/g of the BSG mixture, along with a surfactant (Tween 80), and a laccase enzyme. The mixture was then rested for 48 hours. Following this, the mixture was pumped out of the stirred tank and allowed to flow over mesh sieves (700 μm-100 μm) separating the sugar syrup from the residual high protein mass.

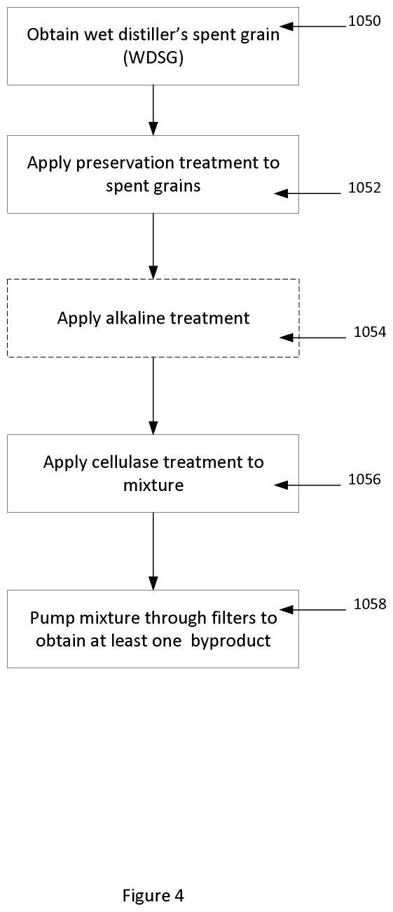

Turning to FIG. 4, a flowchart showing another embodiment of producing at least one byproduct from a spent grain is shown. The current method may be seen as a process of fractionating Wet Distiller's Spent Grain (WDSG).

Initially, the WDSG is obtained from a source (1050). If the distiller's grains is obtained in a dry form, it may be soaked in order to produce the WDSG. The WDSG is then subjected to a preservation treatment (1052). Similar to the process of FIG. 3, the preservation treatment may include any of the treatments listed above. In some embodiments, if the selected preservation treatment is not an alkaline treatment, an alkaline treatment may also be applied (1054).

The preserved WDSG is then subjected to a cellulase treatment (1056) such as those described above with respect to FIG. 3 and then filtered (1058) to separate the at least one byproduct that are produced by the process of FIG. 4. In the current embodiment, the byproducts may be seen as a retentate and a filtrate. For instance, the filtrate may be a sugar syrup while the retentate may be a protein biomass. In one example, a sugar syrup may be separated from the WDSG mixture such as by the use of the progressively fine filtration methods. If desired, or necessary, the residual protein mass may be dried. Again, as with the embodiment of FIG. 3, the filtrate may be discarded after the filtering (1058) and the retentate further processed although the filtrate may also be further processed.

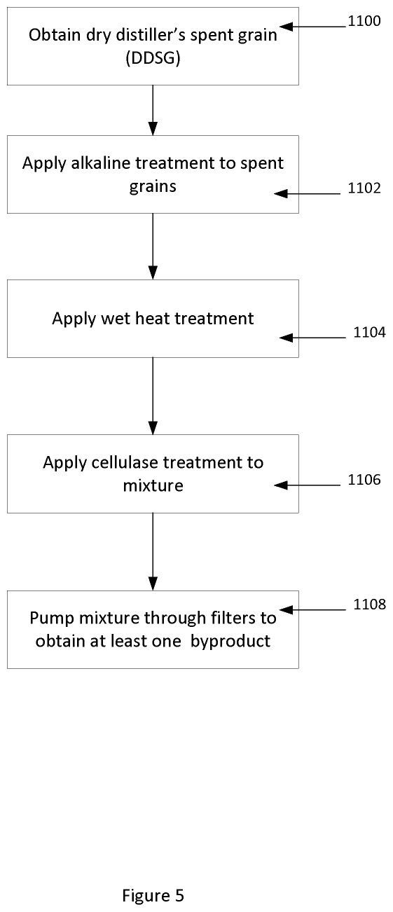

Turning to FIG. 5, a flowchart showing a further embodiment of producing at least one byproduct from a spent grain is shown. The current method may be seen as a process of fractionating Dry Distiller's Spent Grain (DDSG). DDSG may include Dry Distillers Spent Grain with Solubles (DDSGS) or Modified Corn Distillers' Grains with Solubles (MWDSGS).

Initially, the DDSG is obtained from a source (1100). If the distiller's spent grain is obtained in a wet form, it may be placed in an oven to dry it to produce the DDSG.

A preservation treatment is then applied to the DDSG (1102). In one embodiment, the preservation treatment is a treatment of the DDSG with a highly alkaline food grade reagent such as, but not limited to, slaked lime (calcium hydroxide, Ca(OH)2), Soda Ash (sodium carbonate, Na2CO3, washing soda) or caustic soda (sodium hydroxide, NaOH) to a pH>10. After the alkaline treatment, the alkaline DDSG mixture is then exposed to a wet heat treatment (1104) such as, but not limited to, treating the alkaline DDSG mixture with an application of high temperature steam or treating the alkaline DDSG mixture with hot water. In some embodiments, the temperature of the hot water is at a temperature of not less than 50° C.

A cellulase treatment is then applied to the preserved DDG (1106) such as by applying cellulases at defined conditions of temperature and pH for defined time intervals such as disclosed above. The mixture can then be filtered to separate the at least one byproducts from each other (1108) and, if desired or necessary, the at least one byproducts may be dried and further processed, as discussed below. In the current embodiment, the byproducts may be seen as a retentate and a filtrate. For instance the filtrate may be a sugar syrup while the retentate may be a protein biomass. Again, as with the embodiment of FIG. 3, the filtrate may be discarded after the filtering (1108) and the retentate further processed or both may be further processed or only the filtrate processed.

Turning to FIG. 6, a flowchart outlining a method of processing the filtrate, or further processing the at least one byproduct, is shown. After the at least one byproducts have been filtered, a pH of the filtrate (such as the sugar syrup) may be adjusted to be between 3.0 and 4.0 (1150). In one embodiment, this may be performed by treating the filtrate with an acid, such as, but not limited to, a phosphoric acid or citric acid. After the acid treatment, the acid treated filtrate is placed through a separation process (1152). In one embodiment, the separation process may be seen as a process to recover a high protein solution, filtrate or slurry from the acid treated filtrate. The separation process may produce the at least one byproduct, such as a sugar solution and a high protein mass. Examples of separation processes include, but are not limited to, precipitation and decantation, centrifugation or microfiltration. If desired, or necessary, the protein mass may then be dried (1154), such as by tray drying, rotary drying or rolling bed drying to produce a dried protein concentrate.

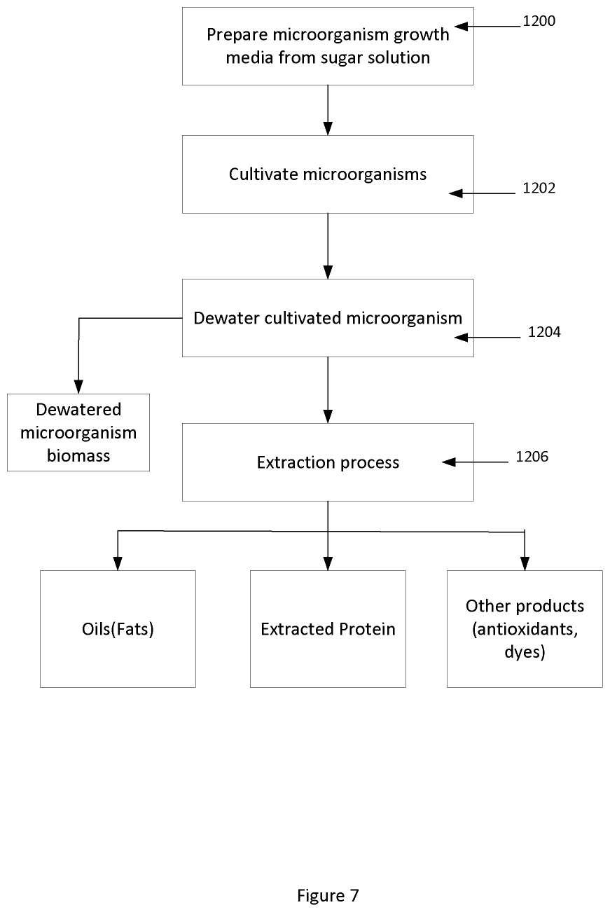

Turning to FIG. 7, a schematic diagram of another embodiment of the further processing of the at least one byproduct is shown. The flowchart of FIG. 7 may be seen as a process for utilizing or processing sugar syrup (or fermentable sugars), or sugar solution, that is produced by processing spent grains. In one embodiment, processing of the fermentable sugars may enable the production of microorganisms, and the extraction of compounds from the cultivated microorganisms.

For example, the fermentable sugars may be used as a carbon source for the heterotrophic cultivation of microalgae, bacteria or eukaryotic microorganisms of various genera. Examples of cultivable microalgae include, but are not restricted to, the genera Chlorella, Schizochytrium and Nanochloropsis. Examples of cultivable bacteria include, but are not limited to, the genera Escherichia, Lactobacillus and Clostridium. Examples of cultivable eukaryotes include, but are not limited to, the genus Aspergillus.

Initially, in this process, suitable microorganism growth media is prepared (1200). In one embodiment, the fermentable sugars or sugar syrup (along with additives supplying macro and micronutrients) is used as a media base for the microorganism growth media. The composition of the media, as well as characteristics such as pH and osmolarity varies are monitored as these characteristics are based on the microorganism being cultivated.

The prepared growth media is then used to cultivate microorganisms in a bioreactor (1202) where the temperature, pH and oxygen levels are carefully controlled. The microorganisms are cultivated until a desired endpoint based on time or biomass concentration is reached. The cultivation process may include one or more phases (nutrient feeding, selective nutrient limitation, continuous cultivation modes). The end of the cultivation corresponds to a process of dewatering (1204) which is performed using techniques including, but not limited to, any combination of settling, flocculation, centrifugation, hot or cold pressing, and filtering. In some embodiments, the dewatered microorganism biomass may be used as a whole cell additive for human and animal nutrition.

In other embodiments, the dewatered cultivated microorganism may undergo an extraction process (1206). The extraction process may include either physical methods, chemical methods, or any combination of the two to extract specific components from the dewatered biomass. Examples of physical extraction methods include, but are not limited to, extrusion and hot and cold pressing. Examples of chemical extraction methods include, but are not limited to, solvent extraction and supercritical carbon dioxide extraction.

The extracted products from the dewatered biomass may include oils and/or fats which may be used in the production of food and cosmetic grade oils. In other embodiments, the extracted product may include an extracted protein that may be used for human nutrition such as for protein powders, textured proteins, or frozen solids and the like. The protein can also be used as additives to various food and beverage products including, but not limited to, shakes, baked goods, formed products, or meat replacement products. In some other embodiments, the extracted product may be used for other products including, but not limited to, antioxidants and dyes.

As one specific example, the sugar solution or syrup is used in place of glucose in a typical fermentation medium such as Proteose Medium (BSG sugar syrup, proteose peptone, NaNO₃, K₂HPO₄, KH₂PO₄, NaCl, CaCl₂-2H₂O, MgSO₄₋₇H₂O). The medium is added to a bioreactor which is then inoculated with a culture of Chlorella vulgaris. The bioreactor is controlled with respect to culture dissolved oxygen and pH level. The fermentation is allowed to run for five days in a fed batch mode, where BSG sugar syrup is used as a supplement. At the end of the fermentation, the generated biomass is settled, a process assisted by a combination of flocculation methods like pH modification and biopolymers such as chitosan. This settled biomass is then subjected to an extraction process such as a combination of extrusion and pressing to release algae oil from the biomass. The oil is further treated to improve clarity, remove waxes, and antioxidants are added. The leftover solid biomass is either converted into a powder, or left as cakes and frozen. This preserved biomass may then be used as an ingredient in meat replacement products.

The residual insoluble high protein mass left after the removal of fermentable sugar syrup has applications in human and animal nutrition. In some embodiments, the residual high protein mass may be used for animal nutrition in the form of powders, pellets or textured protein blocks by any combination of drying, extrusion and pressing. In other embodiments the leftover solid biomass may be used for human nutrition in the form of powders, textured proteins, or frozen solids. This biomass can be incorporated into food and beverage products including, but not limited to: shakes (in the common understanding of the term), baked goods, frozen formed products, meat replacement products such as, but not limited to burgers, sausage patties or nuggets. In another embodiment, the leftover biomass is used as a protein additive for vegan products. In another embodiment, the residual high protein mass is used as an amino acid source in the preparation of fermentation media.

As one specific example, the residual high protein mass is processed by extrusion to form an extruded textured protein product. This protein product can be used as an ingredient in the preparation of meat replacement products such as meat free burgers.

In the preceding description, for purposes of explanation, numerous details are set forth in order to provide a thorough understanding of the embodiments. However, it will be apparent to one skilled in the art that these specific details may not be required. In other instances, well-known structures may be shown in block diagram form in order not to obscure the understanding.

The above-described embodiments are intended to be examples only. Alterations, modifications and variations can be effected to the particular embodiments by those of skill in the art without departing from the scope, which is defined solely by the claims appended hereto.

What is claimed is:

1. A method of producing at least one byproduct from spent grains comprising:
applying a preservation treatment to the spent grains to produce a preserved spent grains mixture;
applying a cellulase treatment to the preserved spent grains mixture to produce a cellulase treated spent grains mixture; and
filtering the cellulase treated spent grains mixture to produce the at least one byproduct.

2. The method of claim 1 further comprising milling the preserved spent grains mixture to reduce a size of particles within the preserved spent grains mixture.

3. The method of claim 2 wherein milling the preserved spent grains mixture comprises wet milling or dry milling the preserved spent grains mixture.

4. The method of claim 1 further comprising wetting the spent grains before applying the preservation treatment.

5. The method of claim 1 wherein applying a preservation treatment comprises:
applying an alkaline treatment to the spent grains.

6. The method of claim 5 wherein applying an alkaline treatment comprises:
applying an alkaline food grade reagent to the spent grains.

7. The method of claim 6 wherein the alkaline food grade reagent comprises slaked lime, soda ash or caustic soda with a pH>10.

8. The method of claim 1 wherein applying a preservation treatment comprises treating the spent grains with a high temperature steam or high temperature water.

9. The method of claim 8 further comprising applying an alkaline treatment after applying the preservation treatment.

10. The method of claim 1 wherein applying a preservation treatment comprises applying a preservative to the spent grains.

11. The method of claim 10 wherein the preservative comprises sodium or potassium metabisulfite or sorbate.

12. The method of claim 1 wherein applying a cellulase treatment comprises:
applying a cellulase to the preserved spent grains mixture at a predetermined temperature and pH level for a predetermined period of time based on the cellulase.

13. The method of claim 12 wherein the cellulase is Cellic Ctec2 or Cellic CTec3.

14. The method of claim 1 wherein filtering the cellulase treated spent grains mixture to produce the at least one byproduct comprises:
filtering the cellulase treated spent grains into a filtrate and a retentate.

15. The method of claim 14 further comprising applying an acid treatment to the filtrate.

16. The method of claim 15 wherein the applying an acid treatment to the filtrate comprises:
adding phosphoric acid or citric acid to the filtrate.

17. The method of claim 14 further comprising:
processing the retentate to produce meal replacement products.

18. The method of claim 14 further comprising:
processing the filtrate to produce at least one of oils, fats, dyes, extracted protein or a dewatered biomass.

19. A system for producing at least one byproduct from spent grains comprising:
a preservation treatment apparatus for receiving the spent grains and for applying a preservative to the spent grains to produce preserved spent grains;
a cellulase treatment apparatus for applying a cellulase treatment to the preserved spent grains to produce cellulase treated spent grains; and
a set of filters for filtering the cellulase treated spent grains.

20. The system of claim 19 further comprising a dry mill or wet mill for miling the preserved spent grains.
